(19) United States
(12) Patent Application Publication
(10) Pub. No.: US 2013/0324985 A1
(43) Pub. Date: Dec. 5, 2013

(54) LASER THERAPY

(71) Applicant: Mark Steven Whiteley, Surrey (GB)

(72) Inventor: Mark Steven Whiteley, Surrey (GB)

(21) Appl. No.: 13/985,667

(22) PCT Filed: Feb. 9, 2012
(86) PCT No.: PCT/GB12/50286

§ 371 Date: Aug. 15, 2013

(30) Foreign Application Priority Data
Feb. 17, 2011 (GB) 1102721.6

(51) Int. Cl. A61B 18/20; A61N 5/06

(52) U.S. Cl. CPC A61B 18/203; A61N 5/0616
USPC 606/9; 607/89

(57) ABSTRACT

The invention relates to a novel medical device for performing laser sweat ablation (LSA) therapy or skin tightening therapy comprising a skin contact member comprising a skin presenting platform of a smooth nature against which skin can smoothly travel and a laser discharge port, which port has a discharge angle between 20° and 60° with respect to the skin contact surface of said platform whereby, in use, laser energy discharging from said port does so at said constant discharge angle whatever the curvature of the skin.

The invention relates to a medical device for performing laser sweat ablation (LSA) or skin tightening surgery and a method of treating hyperhidrosis or skin tone involving the use of said device.

### INTRODUCTION

Hyperhidrosis is a condition that gives rise to excessive perspiration i.e. more than is required for the regulation of body temperature. It is usually classified according to whether it is a congenital or acquired trait. Primary hyperhidrosis starts during adolescence, or even before, and is thought to be inherited as an autosomal dominant genetic trait. Primary hyperhidrosis is estimated to affect 3% of the population and it affects males and females equally. Secondary hyperhidrosis can start at any time and may be due to a disorder of the thyroid or pituitary gland, diabetes mellitus, tumors, gout, menopause, certain drugs, or mercury poisoning. Moreover, hyperhidrosis can either be generalized or localized to specific parts of the body. Hands, feet, armpits, and the groin area are among the most active regions of perspiration due to the relatively high concentration of sweat glands; however, any part of the body may be affected.

The cause of primary hyperhidrosis is unknown, although it is thought to be caused by over activity of the sympathetic nervous system. Indeed, nervousness or excitement can exacerbate the situation for many sufferers. Moreover, whilst other factors can play a role, certain foods and drinks, nicotine, caffeine, and smells can trigger a response.

In the first stage, the condition is treated with over-the-counter anti-perspirants because of their ready availability, however, they rarely produce satisfactory results. Sufferers therefore often move onto prescription-strength antiperspirants. These antiperspirants contain aluminum chloride hexahydrate. Antiperspirants with this component need to be regularly applied but unfortunately the aluminum salts can gather in the sweat ducts and block them. This can lead to skin irritation requiring the use of an anti-inflammatory medication such as hydrocortisone. Iontophoresis is also used to treat the condition. This involves the use of water to conduct an electric current across the skin and so prevent sweat production. Unfortunately this treatment requires periodic repetition. Oral medications are also available which suppress the activity of the sympathetic nervous system but they are not favoured because of their known side effects such as insomnia, blurred vision, and dry mouth. More recently, Botulinum Toxin (or botox) has been used. This procedure involves the injection of botox into the arm pit to reduce underarm sweating. The injections are uncomfortable and have to be repeated when the effect of the toxin wears off. Surgery can also be performed to treat this condition. Endoscopic Thoracic Sympathectomy (ETS) can be undertaken. It involves destroying the supply of nerves to the sweat glands. This is done by inserting an endoscopic instrument into the patient's chest below the armpit. This procedure may be effective but not risk-free. Complications may arise including sweating excessively in other parts of the body as well as nerve problems. Since many of this procedure's complications are serious and irreversible, it is rarely used. Sweat gland removal or destruction may also be undertaken. There are multiple methods for sweat gland removal or destruction such as sweat gland suction, retrodermal currettage, axillary liposuction, subdermal laser ablation or Laser Sweat Ablation.

Laser ablation is the removal of tissue by irradiating it with a laser beam. At low laser flux, the material is heated by the absorbed laser energy and evaporates or sublimates. Usually, laser ablation refers to removing material with a pulsed laser, but it is possible to ablate material with a continuous wave laser beam if the laser intensity is high enough.

The depth over which the laser energy is absorbed and thus the amount of material removed by a single laser pulse depends on several factors including the material's optical properties, the laser wavelength, the angle of incidence of the laser beam to the tissue, the power of the laser pulse and the diameter of the laser fibre.

Laser pulses can vary over a very wide range of duration (milliseconds to femtoseconds) and fluxes, and can be precisely controlled. This makes laser ablation very valuable.

Although laser ablation can be undertaken successfully for the removal of sweat glands or the tightening of skin there are some instances where skin can be damaged or even burned. We have therefore attempted to produce a device that negates the possibility of this happening. Our device controls the angle that the laser subtends the skin even when moving across curved or uneven skin surfaces.

### STATEMENTS OF INVENTION

According to a first aspect of the invention there is provided a medical device for performing laser sweat abalation (LSA) or skin tightening therapy comprising: an elongate member having an inner longitudinal optic guide channel along or through which a selected laser can travel and, at a first end, a skin contact member and, at a second end, a manipulating region; wherein said skin contact member is either fixedly attached to said first end or contiguous therewith and comprises a skin presenting platform of a smooth nature against which skin can smoothly travel and a laser discharge port, in optical communication with said guide channel, which port has a selected discharge angle between 20-60° with respect to the skin contact surface of said platform whereby whatever the curvature of the skin, laser energy is discharged with respect to same at said selected 20-60° angle.

In a preferred embodiment of the invention said angle is between 25-45°, and more ideally between 25-35° and more ideally still selected from one of the following options 25°, 26°, 27°, 28°, 29°, 30°, 31°, 32°, 33°, 34°, or 35°.

In a further preferred embodiment of the invention said platform has curved ends, or is u-shaped, to assist the passage of skin thereover or thereon. Ideally, said platform has a tapered end such that it can easily be inserted into a small skin incision. A further advantage of this tapered structure is that it offers low resistance when travelling against the skin. Ideally said platform is tear-drop shaped. Moreover, said skin contact member or said platform is fixed to said device so that is does not move or rock.

In a further preferred embodiment of the invention said manipulating region may be fashioned to provide a handle.

In yet a further preferred embodiment of the invention said device is provided with an attachment for enabling a laser fibre to be secured in said optic guide channel. This attachment, ideally, comprises a releasable locking mechanism such as a luer lock.

It will apparent to those skilled in the art that the design of the device enables a user to perform LSA or skin tightening by running the device against, i.e. either over or under, a selected area of skin in the sure knowledge that laser light energy emanating from said device will subtend each part of the skin, and so each sweat gland or each part of the dermis, exposed to said energy at a constant angle i.e. the angle of the discharge port with respect to the skin, thus ensuring that the effect of said energy is consistent throughout treatment whatever the nature of the skin terrain.

It will further be apparent to those skilled in the art that the device is used for cosmetic or medical purposes.

According to a further aspect of the invention there is provided a method for performing LSA or skin tightening comprising:
a) placing an laser device against an area of skin to be treated wherein said device comprises an elongate member having, at a first end, a skin contact member comprising a skin presenting platform of a smooth nature against which skin can smoothly travel and a laser discharge port, which port is angled between 20-60° with respect to the skin contact surface of said platform and is in optical communication with an inner longitudinal optic guide channel provided in said device;
b) selecting a laser wave energy for transmitting through said inner longitudinal optic guide and out of said discharge port;
c) moving said device against an area of skin to be treated using a manipulating region provided at a second end of said device whereby;
d) whatever the curvature of the skin, laser energy is discharged with respect to same at said selected angle.

In a preferred method of the invention said device is moved over or under an area of skin to be treated.

In a further preferred method the treatment undertaken is cosmetic.

In a preferred method of the invention said angle is between 25-45°, and more ideally between 25-35° and more ideally still selected from one of the following options 25°, 26°, 27°, 28°, 29°, 30°, 31°, 32°, 33°, 34°, or 35°.

In a further preferred embodiment laser wave energy between 2-20 Watts is used.

Preferably, we pretreat the skin by injecting a local anaesthetic under the skin and then using a blunt dissector to raise the skin to be treated off underlying structures. This allows a working area where it is possible to get to the underside of the skin through one or more tiny incisions. It is then possible to treat the deep dermis using the device of the invention.

Most preferably, said device is used to ablate tissue in the arm pit but it can also be used in other areas where there is a density of sweat glands such as the groin or back. For skin tightening purposes the device may be used on the neck, arms or abdomen.

In yet a further preferred aspect of the invention there is provided a skin contact member for attaching to a laser energy emitting device wherein said member comprises: a skin presenting platform of a smooth nature against which skin can make contact and smoothly travel; and a laser discharge port, which port, in use, is in optic communication with the laser energy of said laser energy emitting device and wherein said discharge port is angled at between 20-60° with respect to the skin contact surface of said platform.

In a preferred embodiment of the invention said angle is between 25-45°, and more ideally between 25-35° and more ideally still selected from one of the following options 25°, 26°, 27°, 28°, 29°, 30°, 31°, 32°, 33°, 34°, or 35°.

Any of the afore aspects of the invention may, in preferred embodiments, include or be characterised by any of the aforementioned features pertaining to the device or the member. Thus, preferred features of each aspect of the invention may be as described in connection with any of the other aspects.

In the claims which follow and in the preceding description of the invention, except where the context requires otherwise due to express language or necessary implication, the word “comprises”, or variations such as “comprised” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The invention will now be described by way of example only with reference to the following figures, wherein:

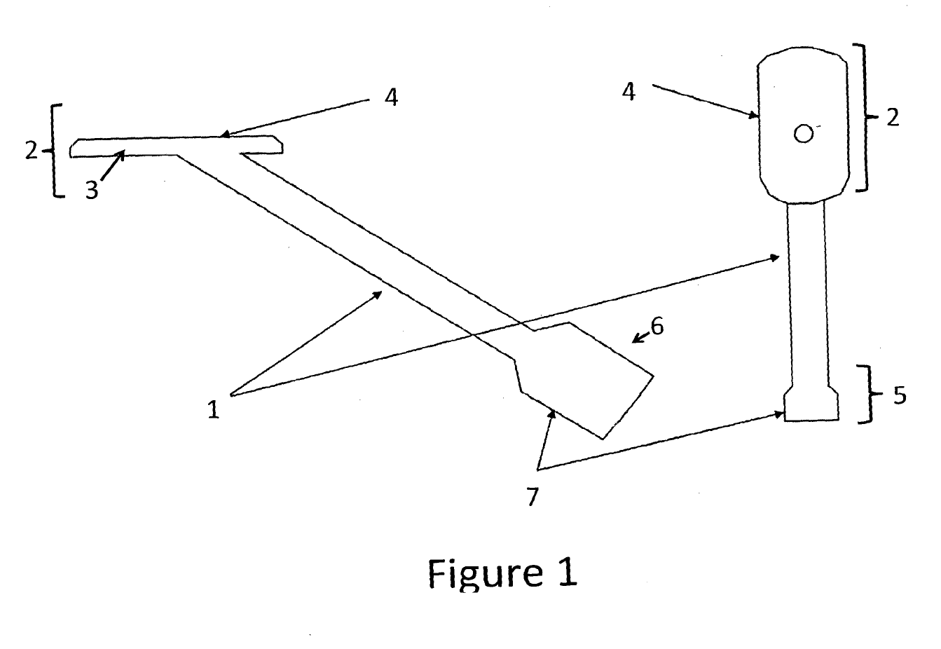

FIG. 1 shows a side elevation view and a plan view of a device in accordance with the invention;

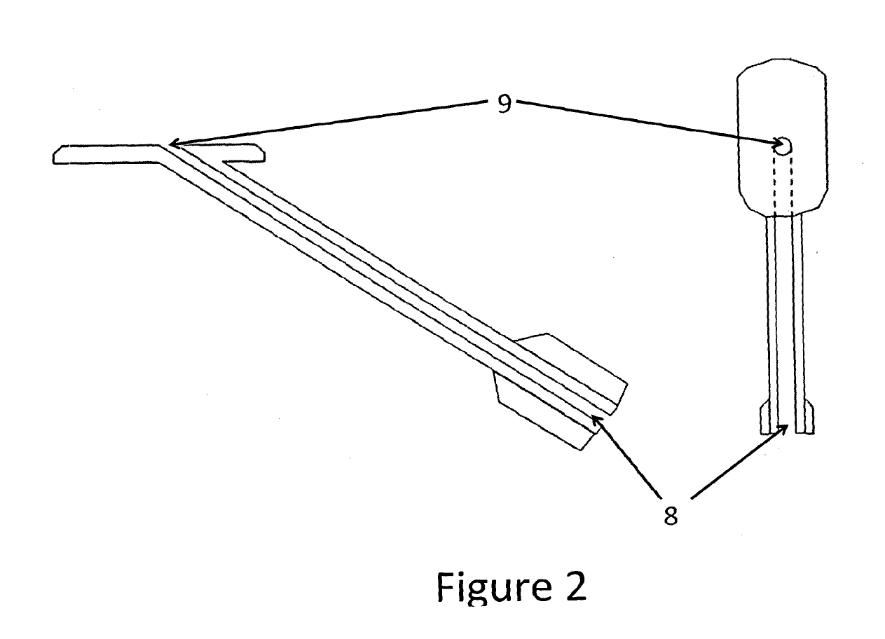

FIG. 2 shows a sectional view of the side elevation and plan views of FIG. 1;

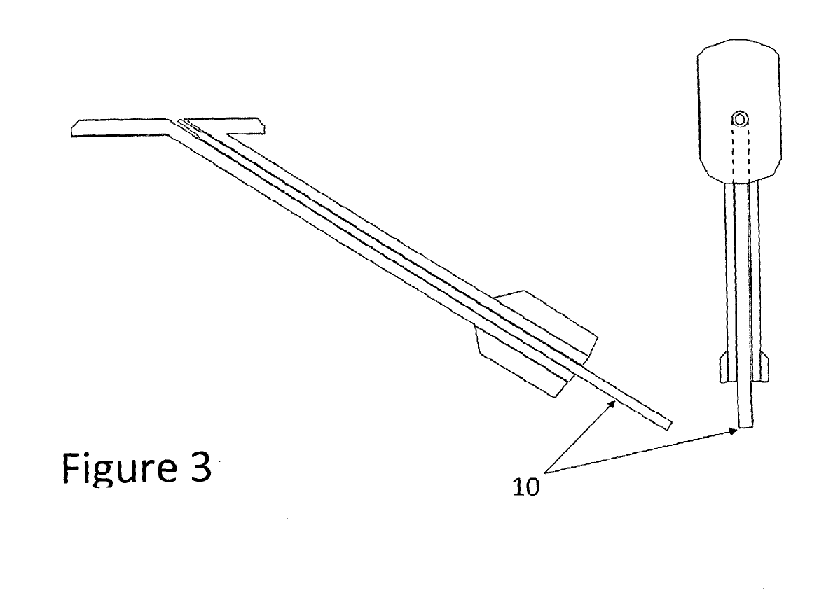

FIG. 3 shows the views of FIG. 2 when used with a laser fibre;

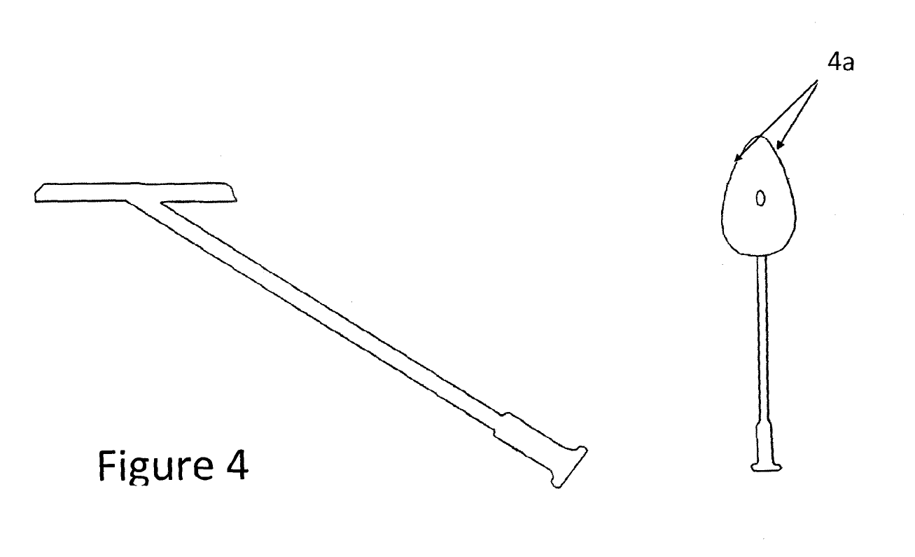

FIG. 4 shows a side elevation view (left hand side) and a plan view (right hand side) of a device in accordance with a further preferred embodiment of the invention;

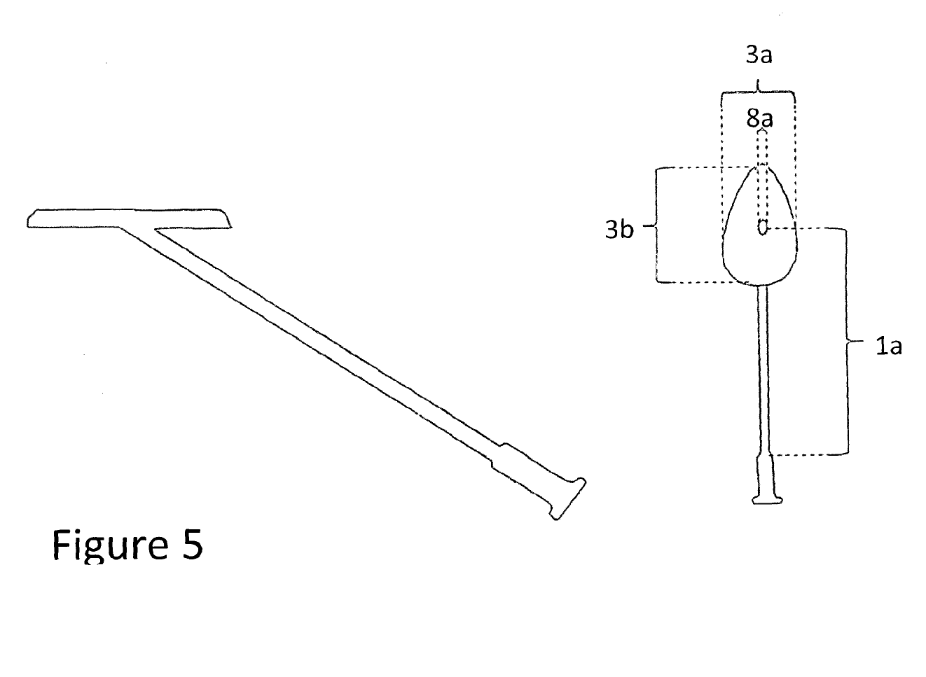

FIG. 5 shows the views of FIG. 4 with preferred dimensions for use indicated using reference numerals; and

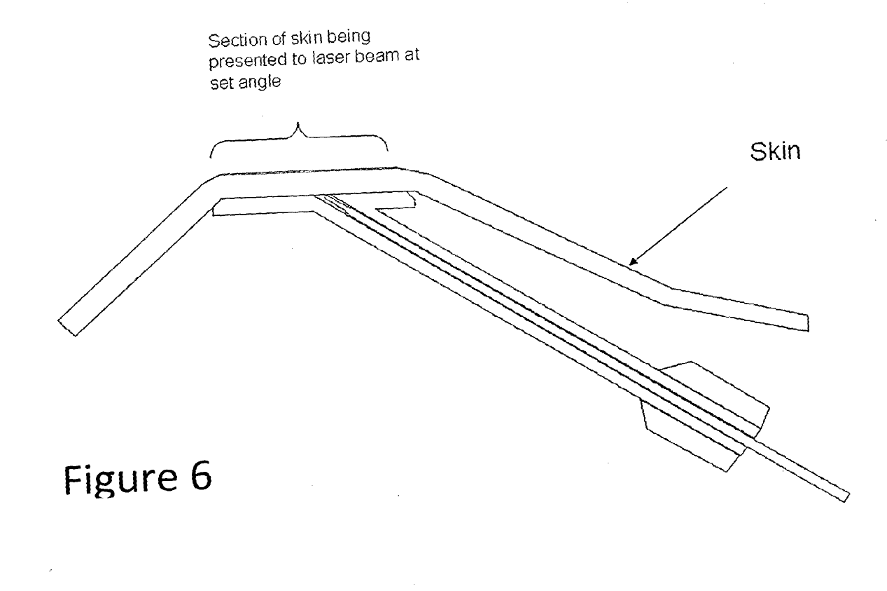

FIG. 6 shows a side sectional view of the device when in use.

Referring to FIG. 1, left hand side, there is shown a side elevation view of a device according to the invention. The device is generally elongate and of a size suitable for use in LSA. It is made from metal or plastics material, whichever is preferred, and may be embellished with instruction indicators, gripping protrusions and the like. On the right hand side of FIG. 1 is shown a plan view of the device.

The device has a longitudinal region (1) which is shown here to be straight but may be curved of about 5-15 cm and typically a diameter of between 1-3 mm so that it can be placed within a minor skin incision when it is to be used under an area of skin to be treated. At a first end (2) there is provided a skin contact member (3) which, in this illustration, is contiguous with longitudinal region (1). However, skin contact member (3) may be separate from region (1) but fixed to it in such a way that it cannot move when in use. Member (3) comprises a skin presenting platform (4). Platform (4) is fashioned to be smooth and further includes curved ends or edges so as to ensure that skin travelling thereagainst does so in a smooth and unagitated manner. The paddle shape of platform (4) is best seen with regard to the plan views of FIGS. 1 and 4 although other shapes of this platform may be used to the same end. At a second end (5) there is provided a manipulating region (6) which is fashioned to include a handle (7).

Referring now to FIG. 2, it can be seen that longitudinal region (1) has a central elongate throughbore (8) which, ideally, is sized and shaped to receive a laser fibre. In this embodiment throughbore (8) also extends into skin contact member (3) and at its end forms a laser discharge port (9) which meets the skin presenting platform (4) at a selected angle between 20 and 60 degrees. Although, between 25-45° is preferred and more ideally an angle of between 25-35° is used such as one of the following options 25°, 26°, 27°, 28°, 29°, 30°, 31°, 32°, 33°, 34°, or 35°.

In other embodiments (not shown) skin contact member (3) may be provided as a separate member that attaches, releasably or otherwise, to region (1) in a manner whereby port (9) is aligned with throughbore (8) to provide a continuous laser delivery channel.

In FIG. 3 the device is shown with a laser fibre (10) extending along throughbore (8) to the laser discharge port (9). Although not illustrated, the laser fibre (10) is held in place using suitable attachments means or a releasable locking system such as a luer lock. However, other sorts of locks may be chosen and are well known to those skilled in the art.

In FIG. 4, left hand side, there is shown a side elevation view of a device according to a further preferred embodiment of the invention. On the right hand side of FIG. 4 is shown a plan view of the device, wherein the preferred design of the skin presenting platform (4) is tear-drop shaped. The inward tapered end 4a permits easy entry into a small skin incision and, further, also offers low resistance travelling against the skin when in use.

Referring to FIG. 5, there is shown the preferred dimensions of the device according to the embodiment depicted in FIG. 4. The central elongate throughbore (8) of longitudinal region (1) is ideally sized and shaped to receive a laser fibre of at least 0.5 mm in diameter (8a). Further, the longitudinal region (1) is ideally approximately 200 mm in length (1a). The skin contact member (3) is fashioned such that it is suited for passage across or under the skin with maximum manoeverability, and ideally is approximately 5 mm in width (3a) and approximately 10 mm in length (3b).

In FIG. 6 there is shown a diagrammatic representation of the device when in use. Typically, when using the device in the armpit region, a small incision is made in the skin of a patient. The incision is of a size suitable for inserting the first end (2) of the device under the skin. Thereafter the skin contact member (3) is adjusted so that the skin presenting platform (4) makes contact with the under side of the skin. The laser is then activated and laser energy exits from discharge port (9) at a fixed angle with respect to both the skin presenting platform (4) and the skin lying thereon or thereagainst. Using handle (7) the device is then pulled along the skin underside so that laser energy is directed at the sub epidermal tissue bearing the sweat glands. For successful ablation we use a laser having the following specification: Fotona XP2 Nd:YAG set at 10 watts with a 40 Hz quasi continuous-wave.

Use of the device as afore described thus ensures that the requisite laser energy is directed towards target tissue in a consistent way throughout use of the device or as the device travels under (or over) skin tissue.

1. A device for performing laser sweat ablation (LSA) or skin tightening therapy comprising: an elongate member having an inner longitudinal optic guide channel along or through which a selected laser energy can travel and, at a first end, a skin contact member and, at a second end, a manipulating region; wherein said skin contact member is either fixedly attached to said first end or contiguous therewith and comprises a skin presenting platform of a smooth nature against which skin can smoothly travel and a laser discharge port, in optical communication with said guide channel, which port has a selected discharge angle between 20-60° with respect to the skin contact surface of said platform whereby whatever the curvature of the skin, said selected laser energy is discharged with respect to same at said selected 20-60° angle.

2. The device according to claim 1 wherein said platform has curved edges or ends to assist the passage of skin thereover or thereon.

3. The device according to claim 1 wherein said platform is tear-drop shaped.

4. The device according to claim 1 wherein said skin contact member or said platform is fixed to said device so that it does not move or rock.

5. The device according to claim 1 wherein said manipulating region may be fashioned to provide a handle.

6. The device according to claim 1 wherein said device is provided with an attachment for enabling a laser fibre to be secured in said optic guide channel.

7. The device according to claim 6 wherein the attachment comprises a releasable locking mechanism.

8. The device according to claim 1, wherein said angle is selected from the group consisting of 25°, 26°, 27°, 28°, 29°, 30°, 31°, 32°, 33°, 34°, and 35°.

9. A method for performing laser sweat ablation (LSA) comprising:
a) placing a laser device against an area of skin to be treated wherein said device comprises an elongate member having, at a first end, a skin contact member comprising a skin presenting platform of a smooth nature against which skin can smoothly travel and a laser discharge port, which port has a selected discharge angle of between 20-60° with respect to the skin contact surface of said platform and is in optical communication with an inner longitudinal optic guide channel provided in said device;
b) selecting a laser wave energy for transmitting through said inner longitudinal optic guide and out of said discharge port;
c) moving said device against an area of skin to be treated using a manipulating region provided at a second end of said device whereby whatever the curvature of the skin, said laser wave energy is discharged with respect to same at said selected angle
a) .

10. The method according to claim 9 wherein said device is moved over or under an area of skin to be treated.

11. The method according to claim 9 wherein said angle is selected from the group consisting of 25°, 26°, 27°, 28°, 29°, 30°, 31°, 32°, 33°, 34°, and 35°.

12. The device according to claim 1, wherein the laser sweat ablation or skin tightening therapy is performed to ablate tissue in the arm pit or groin, or to tighten skin in the neck, arms or abdomen.

13. A skin contact member for attaching to a laser energy emitting device wherein said member comprises: a skin presenting platform of a smooth nature against which skin can make contact and smoothly travel; and a laser discharge port, which port, in use, is in optic communication with the laser energy of said laser energy emitting device and wherein said discharge port is angled between 20-60° with respect to the skin contact surface of said platform.
